(19) United States
(12) Patent Application Publication
(10) Pub. No.: US 2014/0116119 A1
(43) Pub. Date: May 1, 2014

(54) Device and Method for Measuring a Quantity of Water in Oil or Oil Products

(71) Applicants: Peter Mouler, Irvine, CA (US); Andrei Kaloshin, Moscow (RU); Roman Danilov, Moscow (RU); Nikolai Evstigneev, Moscow (RU)

(72) Inventors: Peter Mouler, Irvine, CA (US); Andrei Kaloshin, Moscow (RU); Roman Danilov, Moscow (RU); Nikolai Evstigneev, Moscow (RU)

(73) Assignee: Argosy Technologies, Palm Desert, CA (US)

(21) Appl. No.: 13/660,142

(22) Filed: Oct. 25, 2012

(51) Int. Cl. G01N 33/28

(52) U.S. Cl. CPC G01N 33/28
USPC 73/61.61

(57) ABSTRACT

For measuring a quantity of water on oil and oil products, a hollow coaxial resonator unit is provided, through which oil or oil product is supplied to pass through, an electrical measuring bridge composed of electrical resistances is arranged so that the resonator is connected with the electrical measuring bridge; changes in a frequency characteristic of the resonator are measured when oil or oil product passes through it, and a quantity of water in the oil or oil product is determined based on the measured changes in the frequency characteristic of the resonator.

### BACKGROUND OF THE INVENTION

The present invention relates to devices and method for determination of quantity of moisture or water in oil and oil products.

It is well know that in oil and oil products industries it is important to determine a quantity of water in oil and oil products. Numerous devices and methods have been developed in the prior art and used in practice. One of such device is disclosed for example in Utility Model of Russian Federation RU 94,344. It is believed that the existing devices and methods for measuring a quantity of water in oil and oil products should be further improved to increase the accuracy of measurements and measuring results.

### SUMMARY OF THE INVENTION

Accordingly, it is an object of the present invention to provide a device and a method of measuring a quantity of water in oil and oil products, which have further improvements over the existing devices and methods of this type.

In keeping with these objects and with others which will become apparent hereinafter, one feature of the present invention resides, briefly stated in a device for measuring a quantity of water on oil and oil products, which has a hollow coaxial resonator unit, through which oil or oil product is supplied to pass through, an electrical measuring bridge composed of electrical resistances and arranged so that said resonator is connected with said electrical measuring bridge, means for measuring changes in a frequency characteristic of said resonator when oil or oil product passes through it, and means for determining a quantity of water in the oil or oil product based on the measured changes in the frequency characteristic of said resonator.

The present invention also resides in a method for measuring a quantity of water on oil and oil products, which includes supplying oil or oil product through a hollow coaxial resonator unit, so that oil or oil product passes through the latter, connecting an electrical measuring bridge composed of electrical resistances with said hollow coaxial resonator unit, measuring changes in a frequency characteristic of said resonator when oil or oil product passes through it by measuring means, and determining a quantity of water in the oil or oil product based on the measured changes in the frequency characteristic of said resonator by determining means.

In the inventive device and method the measuring a quantity of water in oil and oil products can include the measuring of the frequency characteristic of said resonator within a range of frequencies.

Also the inventive method and device can include the use of a synthesizer of high frequency oscillations connected with said electrical measuring bridge, a quarts resonator, a detector of high frequency voltage, a differential amplifier, an analog-digital converter, a microcontroller, and interface circuits.

In accordance with the invention, the characteristic of said hollow coaxial resonator can be corrected due to temperature by a temperature sensor. Furthermore, approximations of measurement results can be provided with a plane. in accordance with density, in accordance with saltiness.

The novel features of the present invention are set forth in particular in the appended claims.

The invention itself however will be best understood from the following description of preferred embodiments, which is accompanied by the following drawings

### BRIEF DESCRIPTION OF THE DRAWINGS

FIG. 1 is a view showing a construction of a device for measuring a quantity of water in oil and oil products, with its electrical and electronic components, in which a method for measuring a quantity of water in oil and oil product can be implemented;

FIG. 2 is a view showing frequency characteristics of a hollow coaxial resonator unit of the inventive device for measuring a quantity of water in oil and oil products;

FIG. 3 is a view illustrating a mode for resonators of the inventive device, used for oil and oil products with a low quantity of water in oil and oil products; and

FIG. 4 is a view illustrating a mode for resonators of the inventive device, used for oil and oil products with a high quantity of water in oil and oil products.

### DESCRIPTION OF THE PREFERRED EMBODIMENTS

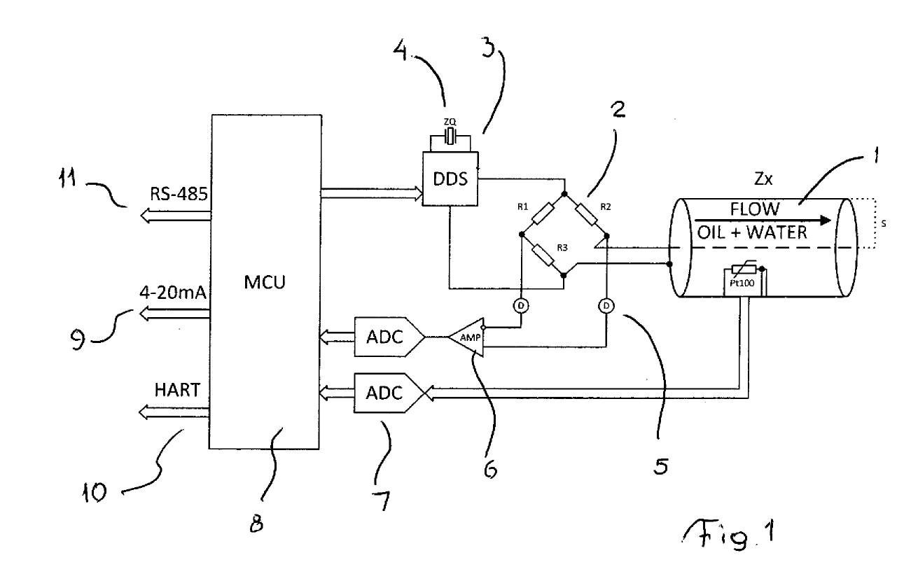

A device for measuring a quantity of water in oil and oil products in accordance with the present invention is shown in detail in FIG. 1. The inventive device has a hollow coaxial resonator Zx which is identified with reference numeral 1. An electrical measuring bridge 2 is connected with the resonator 1 is composed of resistances R1, R2, R3. A synthesizer DDS of high frequency oscillations 3 can be for example a generator AD9912 of Analog Devices. A quarts generator ZQ is identified with 4 and connected with the synthesizer 3. Integral detectors D of high frequency 5, for example of Analog Devices, are connected with the electrical measuring bridge 2 and with an integral circuit AMP of operational amplifier, for example of Analog Devices, identified with reference numeral 6. Analog—digital convertors ADC identified as 7 are connected to a microcontroller MCU identified as 8. Reference numeral 9 identifies a circuit of former of standard current signal from 4 to 20 mA, Reference numeral 10 identifies a circuit of former of signal according to standard HART. Reference numeral 11 identifies a circuit of former of convertor of a digital signal into a signal according to standard RS-485. Finally platinum thermal resistor or sensor PT100, for example by Ruger, is identified as 12.

The device in accordance with the present invention provides actually measurement of disbalance of the electrical measuring bridge 2 in dependence of frequency. The frequency is provided on command from the microcontroller 8, the resistance R1-R2-R3 is constant, the resistance Zx depends on geometrical dimensions of the resonator 1 and on properties of liquid, including water, or in other words depends on a value to be measured.

The synthesizer 3 forms a high frequency voltage for excitation of the measuring bridge 2. The frequency of oscillations if determined by the frequency of the quarts generator 4 and condition of frequency tuning word (FTW) which is provided by the command of the microcontroller. Voltage of disbalance of the measuring bridge 2 depends on resistance Zx and is measured by the detectors 5. For example, if Zx=R the disbalance is equal zero. In order to increase the resolution and sensitivity of the device. the value of the disbalance, converted by the detectors into direct current voltage is amplified by the amplifier 6. The amplified voltage of disbalance is converted into a digital code by the analog-digital convertor 7 and read by the microcontroller 8. Thereby, in the memory of the microcontroller a series of measurements, obtained from response of the measuring bridge on given frequencies of the generator is formed. For a temperature correction, the temperature of luiquid passing through the resonator 1 is measured by a resistor Pt100, the value of resistance is measured by the analog-digital convertor and read by the microprocessor. The microcircuits RS-485, 4-20 mA and HART provide formation of standard interface data transmission

In operation an oil-water mixture is introduced into an interior of the hollow coaxial resonator 1, connected to the measuring bridge 2 and formed by a pipe and antenna, and changes its complex resistance and frequency characteristic. The electronic circuit including the synthesizer 3, the detectors 5 with the amplifier 6 and the analog-digital convertor 6, with the control by the microcontroller 8 measures the frequency characteristic of the resonator 1, the thermosensor 12 and the analog-digital convertor measure temperature of the liquid, the microcontroller 8 based on the frequency characteristic calculates a useful parameter of the characteristic, then the values of the parameter, temperature and calibration coefficients which are determined experimentally for the particular resonator are introduced into a formula for calculation of content of water in oil or oil product. The calculated value of water content in oil or oil product is converted into an analog signal 4-20 Ma and written into memory cells of the microcontroller 8, which are accessible via the interfaces RS-485 and HART.

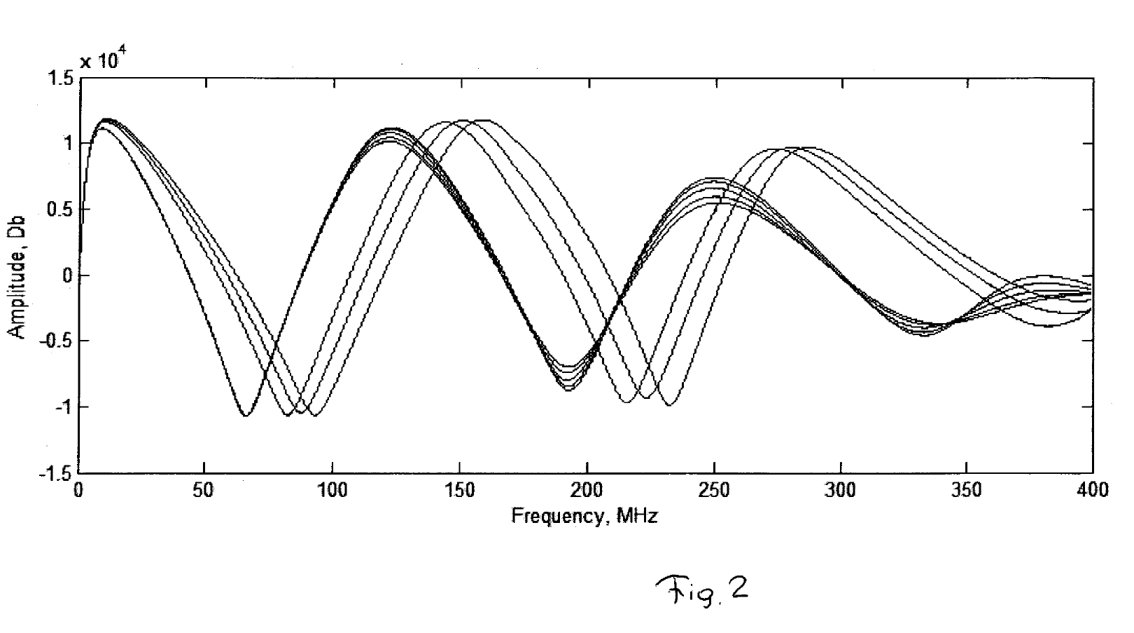

Typical frequency characteristics are shown in FIG. 2. It illustrates dependency of the disbalance of the measuring bridge from the frequency. Several curves shown in this Figure represent dependency of a characteristic from a water content and temperature.

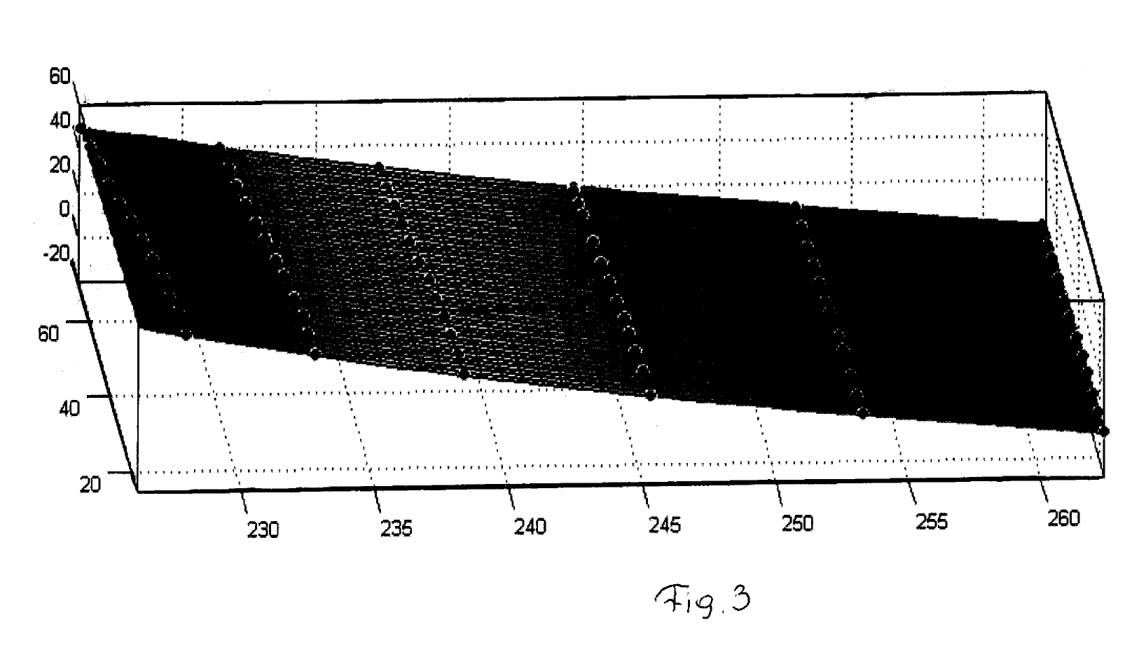

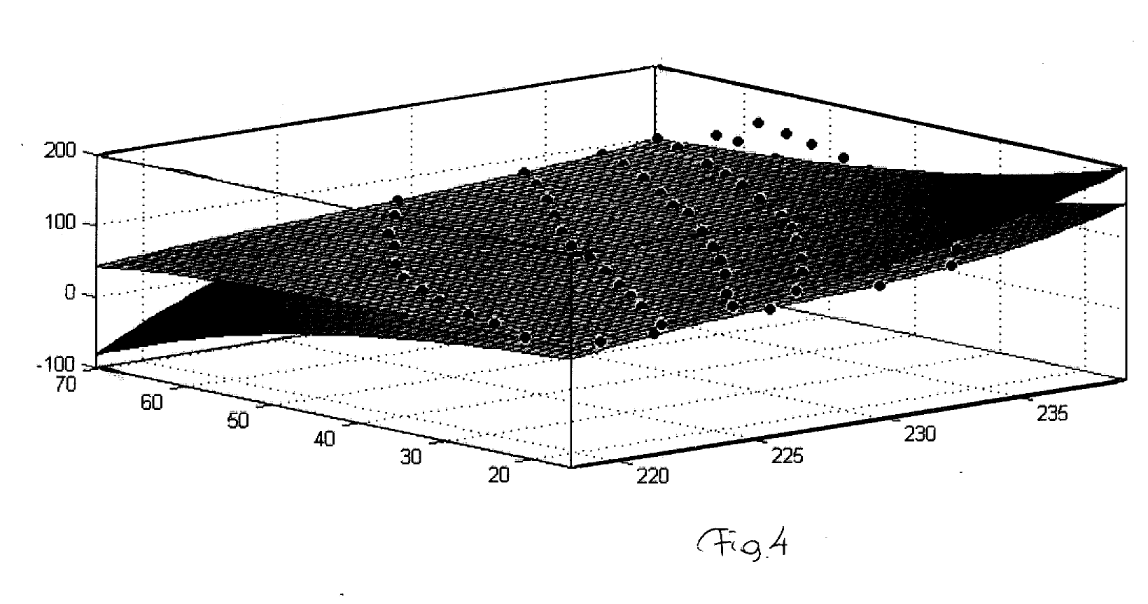

FIGS. 3 and 4 illustrate mode foe resonators filled with a mixture having a low water quantity (from 0% to 50%) and high water quantity (from 50% to 100%).

With the first mentioned mode a scanning algorythm is used, selecting a frequency with which a balancing of the measuring bridge takes place. With the use of coaxial closed resonators with diameter 50-10 mm and length about 1 m there are up to 5 frequencies of balancing of the bridge. As a working parameter, a frequency corresponding to a standing wave of voltage is selected. The dependency of the parameter from the temperature is shown in FIG. 3.

With the second mentioned mode changes in water content in the mixture lead to changes of an active component of the complex resistance of the resonator and small change of length of the standing wave. The sensitivity of the first mode which uses as a working parameter the frequency of balancing of the bridge therefore reduces many times. In the second mode an algorithm of statistic analysis of the characteristic is used, which allows to calculate the parameter of the characteristic. The calculations of water content is carried out in accordance with the statistic parameter. FIG. 4 shows dependency of the parameter from content of water in the mixture, temperature for two different saltiness of water.

The invention is not limited to the details shown since various modifications and structural changes are possible without departing from the spirit of the invention.

What is desired to be protected by letters patent is set forth in the appended claims.

1. A device for measuring a quantity of water on oil and oil products, comprising
a hollow coaxial resonator unit, through which oil or oil product is supplied to pass through;
an electrical measuring bridge composed of electrical resistances and arranged so that said resonator is connected with said electrical measuring bridge;
means for measuring changes in a frequency characteristic of said resonator when oil or oil product passes through it; and
means for determining a quantity of water in the oil or oil product based on the measured changes in the frequency characteristic of said resonator.

2. A device as defined in claim 1, wherein said measuring means is configured as means measuring the frequency characteristic of said resonator within a range of frequencies.

3. A device as defined in claim 1, further comprising a synthesizer of high frequency oscillations connected with said electrical measuring bridge, a quarts resonator, a detector of high frequency voltage, a differential amplifier, an analog-digital converter, a microcontroller, and interface circuits.

4. A device as defined in claim 1, further comprising a temperature sensor configured for correction of the characteristic of said resonator due to temperature.

5. A device as defined in claim 1, further comprising means for providing approximation of measurement results with a plane.

6. A device as defined in claim 1, further comprising means for providing approximation of measurement results in accordance with density.

7. A device as defined in claim 1, further comprising means for providing approximation of measurement results in accordance with saltiness.

8. A method for measuring a quantity of water on oil and oil products, comprising the steps of
supplying oil or oil product through a hollow coaxial resonator unit, so that oil or oil product passes through the latter;
connecting an electrical measuring bridge composed of electrical resistances with said hollow coaxial resonator unit;
measuring changes in a frequency characteristic of said resonator when oil or oil product passes through it by measuring means; and
and determining a quantity of water in the oil or oil product based on the measured changes in the frequency characteristic of said resonator by determining means.

9. A method as defined in claim 8, wherein said measuring includes measuring the frequency characteristic of said resonator within a range of frequencies.

10. A method as defined in claim 1, further comprising providing a synthesizer of high frequency oscillations connected with said electrical measuring bridge, a quarts resonator, a detector of high frequency voltage, a differential amplifier, an analog-digital converter, a microcontroller, and interface circuits.

11. A method as defined in claim 7, further correcting the characteristic of said hollow coaxial resonator due to temperature by a temperature sensor.

12. A method as defined in claim 1, further comprising providing approximation of measurement results with a plane.

13. A method as defined in claim 1, further comprising providing approximation of measurement results in accordance with density.

14. A method as defined in claim 1, further comprising means providing approximation of measurement results in accordance with saltiness.
